(19) United States
(12) Patent Application Publication
(10) Pub. No.: US 2005/0215954 A1
(43) Pub. Date: Sep. 29, 2005

(54) Apparatus and method for maintaining suspendible agents in suspension

(75) Inventor: Frank M. Fago, Mason, OH (US)

Correspondence Address: WOOD, HERRON & EVANS, LLP, 2700 CAREW TOWER, 441 VINE STREET, CINCINNATI, OH, 45202, US

(73) Assignee: Mallinckrodt Inc.

(21) Appl. No.: 10/812,041

(22) Filed: Mar. 29, 2004

(51) Int. Cl. A61M 5/00

(52) U.S. Cl. 604/187

(57) ABSTRACT

An apparatus for maintaining a suspendible contrast agent in suspension in a delivery container before and during administration to a patient. In one embodiment, the suspension apparatus includes a plurality of circumferential flow channels interconnected by radial flow channels. Contrast agent flows through radial flow channels between adjacent circumferential flow channels. Axial flow channels permit the contrast agent to flow, when the delivery container is actuated, between adjacent sets of circumferential flow channels in the apparatus.

### FIELD OF THE INVENTION

The invention relates generally to an apparatus and method for administering an agent and, more particularly, an apparatus and method for administering a volume of a suspended agent to a patient without additional mixing or agitation.

### BACKGROUND OF THE INVENTION

Agents that do not persist in a suspended state within their carrier liquid are typically resuspended before use. Exemplary of such agents are pharmaceutical colloids, including but not limited to contrast agents containing microbubbles, administered or injected into patients for purposes of enhancing images obtained by ultrasound imaging. Contrast agents deliver their maximum effectiveness in image enhancement if uniformly suspended in their fluid base over the duration of the injection. After a syringe is filled with a volume of contrast agent, subsequent delays experienced before injection, such as patient or equipment preparation, and during injection, such as arising from lengthy injection duration, may require agent resuspension.

Contrast agents statically confined in a container without constant or intermittent agitation tend to sediment. For example, microbubbles tend to congregate and agglomerate together due to their inherent buoyancy in the carrier fluid. Accordingly, contrast agents are routinely resuspended by mechanical agitation by hand or by using a mechanical mixing device in advance of use. The pause for resuspending the contrast agent may delay a critical infusion time or, if remixing is omitted, the entire imaging procedure may have to be repeated due to suboptimal contrast obtained. Duplicate procedures not only put patients at increased risk and inconvenience, but are also expensive and time-inefficient. Even if the need to resuspend a single bolus injection is not prohibitive for a given procedure, repeated bolus injections or long term continuous infusions can become problematic due to loss of contrast agent suspension during administration.

It would be desirable, therefore, to provide an apparatus for maintaining a contrast agent in suspension so that the contrast agent may be administered to a patient without additional mixing or agitation.

### SUMMARY

The invention provides an apparatus for maintaining a suspendible contrast agent in suspension pending administration to a patient. The apparatus includes a delivery container with a fluid reservoir capable of holding a propellant fluid, an exit port, a delivery mechanism coupled with the reservoir, and a suspension apparatus positioned inside the delivery container in a fluid path between the fluid reservoir and the port. The suspension apparatus includes a plurality of circumferential flow channels and at least one radial flow channel capable of being filled with the contrast agent. Adjacent circumferential flow channels are coupled in fluid communication by a radial flow channel and the circumferential and radial flow channels are coupled in fluid communication with the exit port. The contrast agent is delivered to the exit port after flowing through the radial flow channel and the plurality of circumferential flow channels when the delivery mechanism is operated to cause propellant fluid to flow through the fluid path.

In another embodiment, an apparatus for administering a contrast agent includes a delivery container including a fluid reservoir capable of holding a propellant fluid, an exit port, a delivery mechanism coupled with the reservoir, and a suspension apparatus positioned inside the delivery container in a fluid path between the fluid reservoir and the exit port. The suspension apparatus includes a plurality of first and second plates in a stacked arrangement. Each pair of the first and second plates is separated by a plurality of dividing walls defining a plurality of circumferential flow channels capable of being filled with contrast agent. Each of the plurality of first and second plates is configured to permit axial flow between the plurality of circumferential flow channels of adjacent pairs of first and second plates. Contrast agent is delivered to the exit port after flowing through the plurality of circumferential flow channels when the delivery mechanism is operated to cause propellant fluid to flow through the fluid path.

The apparatus of the invention sub-divides a desired volume of an agent and confines sub-volumes of the agent in restricted-volume spaces to maintain agent suspension during storage and/or pending administration to a patient. As a result, the agent may be administered without additional mechanical mixing or agitation, either manually or with a powered appliance. As will be described, the apparatus may be located in the same container, such as a syringe, that contains a propellant fluid used to eject the agent from the apparatus of the invention. Alternatively, the apparatus may be located adjacent an exit port of a propellant fluid container or may be positioned in-line at any point in a fluid path between a propellant fluid container and a patient.

The apparatus passively confines the contrast agent in a static state that reduces settling or sedimentation and enhances suspension without the need for mechanical mixing or suspending before use. Hence, the apparatus of the invention is effective for reducing or eliminating the difficulties associated with remixing or resuspending an agent that has or may have come out of suspension before use. Use may be either readying for an injection by transferring agent from a bulk container to a delivery container such as syringe, or injecting agent from the delivery container into a patient, such as an infusion process. This increases the quality, safety, and time-efficiency of imaging procedures and reduces the procedure costs.

Maintaining the contrast agent in a substantially fully suspendible state assures consistent quality and reduced sensitivity to user technique, because the agent may be shipped already prefilled inside the apparatus. The prefilled configuration has the potential to reduce susceptibility of certain contrast agents to mechanical vibration and shock.

The features and objectives of the invention will become more readily apparent from the following Detailed Description taken in conjunction with the accompanying drawings.

### BRIEF DESCRIPTION OF THE FIGURES

The accompanying drawings, which are incorporated in and constitute a part of this specification, illustrate embodiments of the invention and, together with a general description of the invention given above, and the detailed description given below, serve to explain the principles of the invention.

FIG. 1 is a perspective view of a suspension apparatus of one embodiment of the invention while being filled with contrast agent;

FIG. 2 is a perspective view of the suspension apparatus of FIG. 1 shown with the syringe plunger removed from the apparatus and with the contrast agent omitted for clarity;

FIG. 3 is a partially exploded view of an unfilled suspension apparatus of FIG. 1;

FIGS. 4A and 4B are top and bottom views, respectively, of a baffle plate utilized in the suspension apparatus of FIG. 3;

FIG. 5 is a cross-sectional view taken along line 5-5 of FIG. 2;

FIG. 6 is a top view of an alternative embodiment of the baffle plate in accordance with the principles of the invention;

FIG. 7 is a view of a suspension apparatus in accordance with an alternative embodiment of the invention;

FIG. 8 is a view of a suspension apparatus in accordance with an alternative embodiment of the invention;

FIGS. 8A and 8B are top and bottom views, respectively, of a baffle plate present in the suspension apparatus of FIG. 8;

FIGS. 8C and 8D are top and bottom views, respectively, of a baffle plate used in combination with the baffle plate shown in FIGS. 8A and 8B to construct the suspension apparatus of FIG. 8; and

FIG. 9 is a perspective view shown partially broken away of an alternative embodiment of the invention.

### DETAILED DESCRIPTION

The invention relates to an apparatus and method for maintaining a suspendible agent, such as a contrast agent, in suspension, thus enhancing the agent's effectiveness in an imaging procedure. One type of contrast agent is a suspension of microbubbles to enhance ultrasound imaging. The invention is, however, not limited to particular types of contrast agents to be stored and dispensed, and may be used for storing and dispensing any suspended agent that can be placed within a delivery container, such as a syringe. Although the invention will be described herein as being formed using an exemplary delivery container or syringe, it should be understood that modifications to the exemplary delivery container described herein could be made without departing from the intended spirit and scope of the invention.

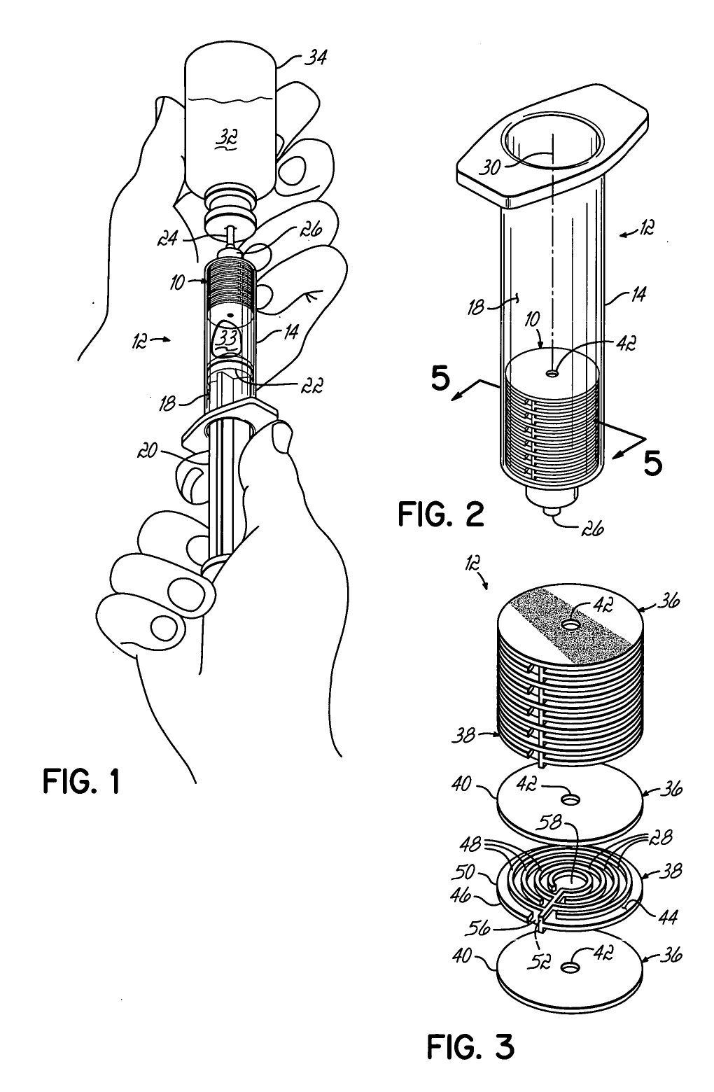

With reference to FIGS. 1 and 2, an insert or suspension apparatus 10 capable of holding a volume of a suspendible contrast agent in a confined space is positioned inside of a delivery container 12. The delivery container 12 includes a barrel 14 having a side wall 16 (FIG. 5) and a plunger 20 featuring a plug 22 having an interference fit with an inner surface 16a of the side wall 16. The volume between the plug 22 and the suspension apparatus 10 defines a variable-volume fluid reservoir 18 (FIG. 2). As the plunger 20 is moved, the volume of the fluid reservoir 18 varies. The interference fit between the plug 22 and side wall 16 is effective for maintaining a seal while simultaneously confining any propellant and/or agent within the fluid reservoir 18.

Although delivery container 12 is depicted in FIGS. 1 and 2 as a conventional syringe and the suspension apparatus 10 is confined inside of delivery container 12, the invention is not so limited. In various alternative embodiments of the invention, suspension apparatus 10 may be deployed inside any suitable delivery container, may be located in an external container adjacent an exit port of a propellant fluid container, or may be positioned in-line at any point in a fluid path extending between a propellant fluid container and a patient. Alternatively, the apparatus 10 may be located adjacent the exit port 26 of an external container.

With continued reference to FIGS. 1 and 2, a standard hypodermic needle 24 is coupled by, for example, a standard luer connection with an exit port 26 of the fluid reservoir 18. When the plunger 20 is withdrawn away from the exit port 26, fluid is aspirated through the hypodermic needle 24 and port 26 into the suspension apparatus 10 and, if the aspiration suffices, through a central opening 42 in the exposed spacer plate 36 (FIG. 3) of suspension apparatus 10 into the fluid reservoir 18. For example, propellant fluid 33 may be aspirated through the suspension apparatus 10 into the volume of the fluid reservoir 18 and/or contrast agent 32 may be aspirated from a bulk container 34 (FIG. 1) into the suspension apparatus 10. The suspension device 10 may be totally or partially filled with contrast agent 32. In the latter circumstance, the unfilled volume of suspension device 10 is filled with propellant fluid.

When the plunger 20 is advanced toward exit port 26, any propellant fluid 33 and/or contrast agent 32 confined inside the fluid reservoir 18 is forced into the suspension apparatus 10, which causes contrast agent 32 and/or propellant fluid 33 confined inside suspension apparatus 10 to be expelled through the exit port 26 and hypodermic needle 24 for administering the agent 32 to a patient. Contrast agent 32 flowing through the suspension device 10 during agent administration remains in a suspended condition or state as spatial confinement is maintained for the duration of the injection. In addition, the loss of suspension of the contrast agent 32 in suspension device 10 is minimized after filling and before agent administration, or under circumstances in which agent administration is interrupted.

With continued reference to FIGS. 3-5, the suspension apparatus 10 is constructed from a plurality of substantially identical spacer plates 36 and a plurality of substantially identical baffle plates 38 arranged in an alternating stacked configuration. The spacer and baffle plates 36, 38 may be fused or otherwise joined together to define an integral unit, or may be discrete components positioned with a contacting arrangement. Each spacer plate 36 is a generally-featureless, smooth-surfaced annular plate having a circular peripheral edge 40 and a central opening 42, which operates as an axial flow channel in suspension apparatus 10. The central openings 42 of the spacer plates 36 may be coaxially aligned with a central longitudinal axis 30 of the barrel 14, although the invention is not so limited. The central opening 42 in the spacer plate 36 adjacent to the fluid reservoir 18 operates as an entry port into the suspension apparatus 10 for propellant fluid 33. The peripheral edge 40 contacts the side wall 16 of the delivery container 12 in a substantially fluid tight manner so that flow is constrained to the central opening 42.

Opposite upstream and downstream surfaces 44, 46 of each baffle plate 38 include a plurality of dividing walls 48 that are arranged spatially on a disk-shaped body 50 and, typically, arranged with a concentric configuration. The open volume between adjacent dividing walls 48 and the open volume between the radially-outermost dividing wall 48 and the side wall 16 of the delivery container 12, which collectively defines circumferential flow channels 28, along with the open volume of the radial flow channels 59 define an open volume in which the contrast agent 32 is stored in a substantially suspended state and ready for immediate administration to a patient without additional agitation. Radially adjacent dividing walls 48 are joined by a length or portion of a radially-extending dividing wall 52 for defining the radial flow channels 59. The suspendible agent experiences a 180 degree change or reversal in fluid flow direction for contrast agent 32 flowing in the flow channels 28 at the dividing wall 52, and flow proceeds at dividing wall 52 radially in the radial flow channels 59.

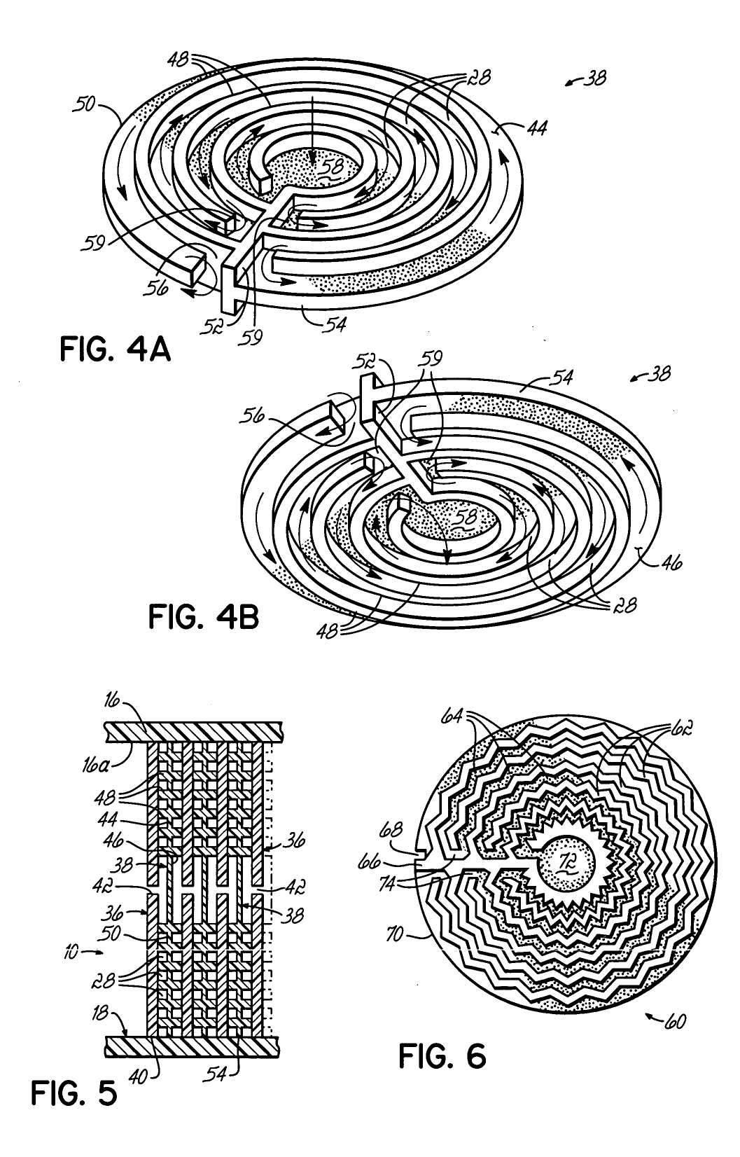

In various embodiments, the baffle plate 38 may be circular, as depicted in FIGS. 4A and 4B, or may possess a different geometrical shape, such as rectangular, to which the side wall 16 is suitably modified in a geometrical shape to be in conformity with the plate 38. The dividing walls 48, 52 may have other tortuous arrangements over the surfaces of baffle plate 38 that provide a continuous fluid path through which the contrast agent 32 is restricted to flow when being administered to a patient.

With continued reference to FIGS. 3-5, one of the spacer plates 36 contacts the dividing walls 48, 52 on each of opposite upstream and downstream surfaces 44, 46 of the baffle plate 38 in a fluid-tight or substantially fluid-tight manner to prevent or limit, respectively, gaps between the spacer plates 36 and baffle plates 38. As a result, the radial agent flow occurs substantially through the radial flow channels 59 and the contrast agent 32 is forced to flow through the convoluted or tortuous fluid path defined by circumferential flow channels 28 when expelled from exit port 26 by advancement of plunger 20 toward port 26.

An axial flow channel 56 permitting flow in a direction parallel to axis 30 is defined between the upstream and downstream surfaces 44, 46 of each baffle plate 38 as a notch or throughhole provided proximate to the peripheral edge 54 of each baffle plate 38. Suspendible agent flows in the axial flow channel 56 between surfaces 44, 46 when the plunger 20 is moved inside barrel 14. A central open space 58 defined proximate the center of the baffle plate 38 is registered with the opening 42 of the adjacent spacer plate 36. Dividing wall 52 obstructs the radially-outermost circumferential flow channel 28 proximate the axial flow channel 56 so that the flowing contrast agent will be directed through the axial flow channel 56 between the opposed upstream and downstream sides of each baffle plate 38.

Suspension apparatus 10 and delivery container 12 are preferably molded from suitable polymers including, but not limited to, polypropylene. The suspension apparatus 10 may also be made from a combination of polymers, rubber or thermoplastic elastomer materials. In certain embodiments of the invention, the ratio of the volume of the channels 28, 56 and 59 (i.e., open space) to the volume occupied by dividing walls 48 and 52 (i.e., filled space) ranges from about 0.25 to about 0.5, which implies in these embodiments that the contrast agent 32 will fill at most 50 percent of the volume of suspension apparatus 10.

In use and with reference to FIGS. 1-5, hypodermic needle 24 of delivery container 12 pierces the septum of a bulk container (not shown) similar to bulk container 34 holding a propellant fluid 33 to establish a fluid connection. The delivery container 12 is shown for simplicity in FIG. 1 in a condition with propellant fluid 33 already filling the fluid reservoir 18. The propellant fluid 33 is any biocompatible viscous fluid and may be a diluent, such as normal saline, water, buffer, etc., for the contrast agent 32. The propellant fluid 33 may also be a second contrast agent having a different composition than the contrast agent 32 injected for the impending imaging procedure. The plunger 20 is moved in a direction away from the exit port 26 by a distance sufficient to aspirate a volume of propellant fluid 33 from a propellant fluid bulk container (not shown) through the flow channels 28, 56 and 59 of the suspension apparatus 10 and into the fluid reservoir 18. Withdrawing the hypodermic needle 24 from the septum of the bulk container terminates the fluid connection.

Next, the septum of the bulk container 34 holding a contrast agent 32 is pierced by hypodermic needle 24 to establish a fluid connection. Again, the plunger 20 is moved in a direction away from the exit port 26 so that a volume of contrast agent 32 is aspirated from the bulk container 34 into the flow channels 28, 56, 59 of the suspension apparatus 10. The contrast agent 32 displaces the propellant fluid 33 in the suspension apparatus 10 from the flow channels 28, 56 and 59 into the fluid reservoir 18 and at least partially fills the flow channels 28, 56 and 59 of the suspension apparatus 10. The aspirated volume of contrast agent 32 may completely or partially fill the suspension apparatus 10, with any unfilled volume occupied by propellant fluid 33. Withdrawing the hypodermic needle 24 from the septum of the bulk container 34 terminates the fluid connection. The invention contemplates that the delivery container 12 may be prefilled with propellant fluid 33 and/or the suspension apparatus 10 prefilled with contrast agent 32 before shipment to an end user.

Exit port 26 of the delivery container 12 is then coupled with a patient for administering the contrast agent inside suspension apparatus 10 to a patient in preparation for an imaging procedure. The contrast agent administered to the patient has been maintained in a substantially suspended state by confinement within the passages of suspension apparatus. The administration may use the original hypodermic needle 24, for example, by connection to a catheter (not shown), or by establishing a different type of fluid connection with the exit port 26. The plunger 20 of delivery container 12 may be advanced manually or using a powered delivery suspension apparatus (not shown) to define a delivery mechanism capable of expelling the contrast agent 32 through the exit port 26. An imaging procedure is conducted while the contrast agent 32 in apparatus 10 is being administered or after the contrast agent 32 in apparatus 10 is administered.

As the plunger 20 is advanced and with particular reference to FIGS. 4A and 4A in which fluid flow in one baffle plate 38 is diagrammatically shown, propellant fluid 33 is forced under pressure from the fluid reservoir 18 into the opening 42 of the baffle plate 38 in the suspension apparatus 10 that opens into the fluid reservoir 18. The pressurized introduction of propellant fluid 33 causes the contrast agent 32 resident in suspension apparatus 10 to flow downstream toward the exit port 26 of the delivery container 12, with which the suspension apparatus 10 is coupled in fluid communication. The contrast agent 32 is constrained to flow in a continuous and serial manner from the central opening 42 in an upstream spacer plate 36, into the center space 58 of the upstream surface 44 of the baffle plate 38, radially outward through the flow channels 28, 59 from the center space 58 to the peripheral edge 54, axially through the axial flow channel 56 to the opposite downstream surface 46 of the baffle plate 38, radially inward through the flow channels 28, 59 defined on the downstream surface 46 of baffle plate 38 to the center space 58 on the downstream surface 46 of the baffle plate 38, and then axially through the central opening 42 in the downstream spacer plate 36 for flow through the channels 28, 56 and 59 of the downstream baffle plate 38. Contrast agent flows through each downstream baffle plate 38 with the same fluid path. The central openings 43 and axial flow channels 56 collectively permit flow of contrast agent 32 in an axial direction toward exit port 26.

The contrast agent 32 is delivered in a substantially suspended state due to the pre-administration confinement in sub-volumes in the set of channels 28, 56, and 59. As the contrast agent 32 is incrementally administered to the patient, the residual volume of contrast agent 32 in the suspension apparatus 10 may be monitored. For example, a user may observe the contrast agent 32 in the radially outermost channel of each set of circumferential flow channels 28 and axial flow channels 56 that are visible through the side wall 16 of delivery container 12. The flow of the suspendible contrast agent 32 through the flow channels 28, 56, and 59 may operate for mixing the agent 32 during administration due to the abrupt 180° reversal of flow direction at the dividing wall 52 and the generally tortuous fluid path defined by the flow channels 28, 56, and 59.

With reference to FIG. 6 and in accordance with an alternative embodiment of the invention, a baffle plate 60 incorporates dividing walls 62 that are undulating in a radial manner. Flow channels 64 are defined between radially-adjacent pairs of the dividing walls 62, and a radially-extending dividing wall 66 blocks each flow channel 64 and defines radial flow channels 74. An axial flow channel 68 is defined by a notch formed in a peripheral edge 70 of the baffle plate 60 and permits flow between the upstream and downstream surfaces, of which only one surface is shown in FIG. 6, of the baffle plate 60. Although only one side of the baffle plate 60 is shown in FIG. 6, it is understood that an identical set of dividing walls (not shown but identical to dividing walls 62) is provided on the opposite surface of the baffle plate 60. A central open space 72 defined proximate the center of the baffle plate 60 is registered with the opening 42 of the adjacent spacer plate 36 (FIG. 3).

The dividing walls 62 incorporate local irregularities, in contrast to the smoothly curving dividing walls 48 of baffle plate 38, causing the contrast agent flowing in channels 64 to experience multiple changes in direction, which is believed to promote turbulent mixing of the contrast agent 32. The turbulent mixing effect supplements the maintained suspension of the contrast agent 32 provided by the spatial confinement within the flow channels 64, 68. The undulations in dividing walls 62 providing the mixing effect are illustrated in FIG. 6 as having a sawtooth appearance with periodic features, although the invention is not limited as other types of wall irregularities capable of mixing the contrast agent 32 are contemplated by the invention. For example, the local irregularities in dividing walls 62 may have a square-wave shape with periodic features having surfaces that intersect at right angles.

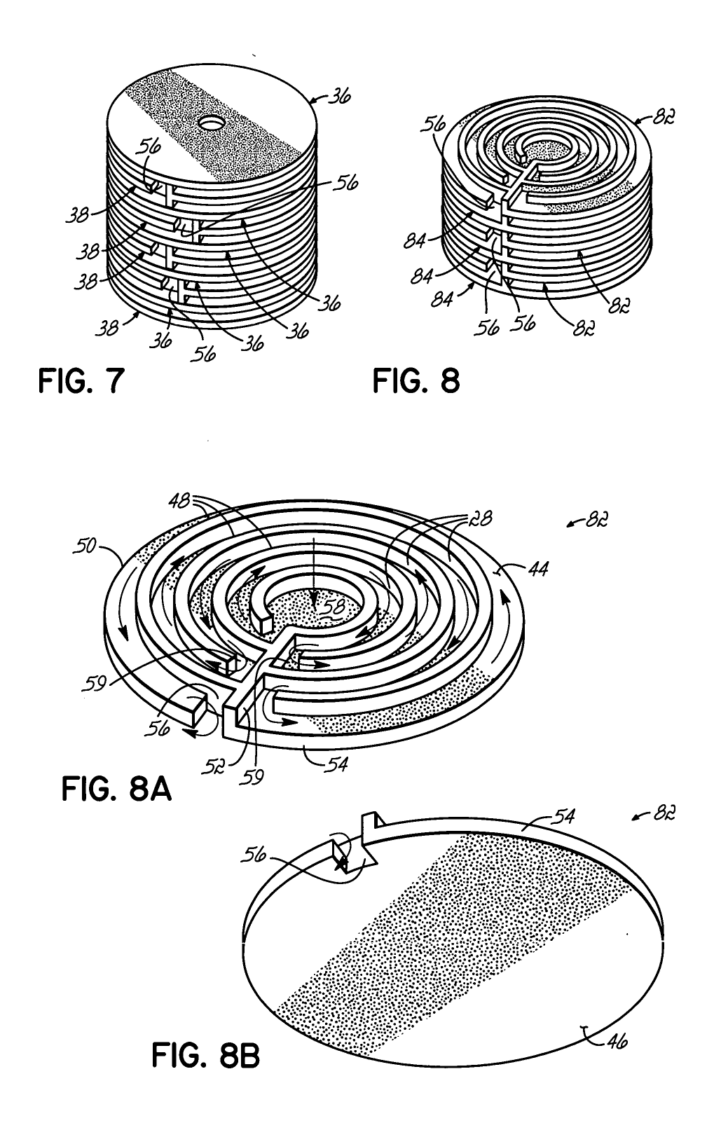

With reference to FIG. 7 in which like reference numerals refer to like features in FIGS. 1-6, the baffle plates 38 of suspension apparatus 10 may be oriented angularly relative to one another such that the axial flow channels 56 are not axially aligned parallel to axis 30, as opposed to the axial alignment depicted in FIGS. 1-6. The angular reorientation is possible because the axial flow through the axial flow channel 56 of each baffle plate 38 is independent of the axial flow of adjacent baffle plates 38.

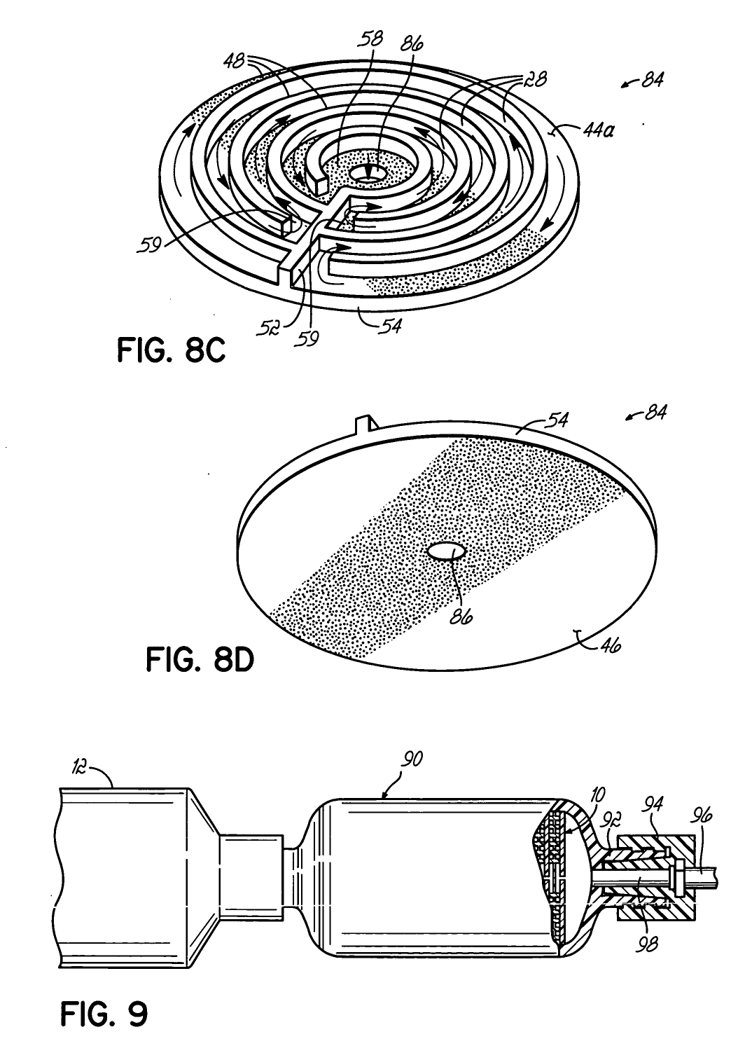

With reference to FIGS. 8 and 8A-D in which like reference numerals refer to like features in FIGS. 1-7 and in accordance with an alternative embodiment of the invention, suspension device 10 may incorporate two different types of baffle plates 82, 84 that are similar to baffle plate 38 and omit the spacer plates 36. This embodiment of the suspension device 10 is assembled by stacking the baffle plates 82, 84 in an alternating manner and in mutual fluid communication inside of delivery container 12. With specific reference to FIGS. 8A and 8B, dividing walls are omitted from the downstream surface 46 of baffle plate 82 so that the downstream surface 46 is featureless. The upstream surface 44 of baffle plate 82 includes dividing walls 48, 52 and the axial flow channel 56 extends axially through body 50. With specific reference to FIGS. 8C and 8D, dividing walls 48, 52 are provided on the upstream surface 44 of baffle plate 84 but the body 50 of baffle plate 84 lacks an axial flow channel adjacent to peripheral edge 54. Instead, baffle plate 84 incorporates a central opening 86 extending through body 50 in center space 56 and aligned substantially coaxial with axis 30 (FIG. 2). The downstream surface 46 of baffle plate 84 lacks dividing walls and, hence, is featureless. The downstream surface 46 of baffle plate 82 is positioned in contact with the dividing walls 48, 52 on the upstream surface 44 of baffle plate 84. If another baffle plate 82 is present, the downstream surface 46 of baffle plate 84 is placed in contact with the dividing walls 48, 52 on the upstream surface 44 of the baffle plate 82. The pattern repeats for additional baffle plates 82, 84.

As the plunger 20 is advanced to administer contrast agent 32 in a suspended condition to a patient as described above, the contrast agent 32 resident in the flow channels 28 and 59 of baffle plates 82, 84, in axial flow 56 of baffle plate 82, and in central opening 86 of baffle plate 84 flows toward the exit port 26 of the delivery container 12 under the pressure provided by propellant fluid 33. More specifically, the contrast agent 32 flows in a continuous and serial manner from the center space 58 on the upstream surface 44 of the baffle plate 82 radially outward through the flow channels 28, 59 to the peripheral edge 54, axially through the axial flow channel 56 to flow channels 28, 59 defined between the downstream surface 46 of baffle plate 82 and the opposite upstream surface 44 of the baffle plate 84, radially inward through the flow channels 28, 59 of baffle plate 84, and axially through the central opening 86 to the center space 58 of the adjacent downstream baffle plate 82.

With reference to FIG. 9 in which like reference numerals refer to like features in FIGS. 1-8, the delivery container 12 further includes an external compartment 90 coupled in fluid communication with the exit port 26 and the suspension apparatus 10 is positioned inside of the external compartment 90. The external compartment 90 may, alternatively, assume the form of a canister (not shown) similar to external compartment 90 that is positioned in-line at any point in a fluid path extending between a propellant fluid container and a patient. A connector 92, such as a male luer fitting, couples the external compartment 90 of delivery container 12 with another connector 94, such as a female luer fitting, on an end of a tube 96 that extends to a patient. The suspension apparatus 10 is coupled in fluid communication with the lumen of the tube 96 by an exit port 98 defined collectively in the external compartment 90 and connector 92.

In another embodiment of the invention, the inventive apparatus and method may also be used to deliver microparticle and/or nanoparticle based contrast agents, as described in U.S. Pat. No. 5,406,950, expressly incorporated by reference herein in its entirety.

In yet another embodiment of the invention, the inventive apparatus and method may also be used to deliver agents for photoacoustic imaging, as described in co-pending U.S. patent application Ser. No. 09/978,725, expressly incorporated by reference herein in its entirety. For example, an optical imaging agent may be incorporated into a microbubble-containing ultrasound contrast agent and administered as described. Optical tomography excites and detects light at selected wavelengths for an optical image, and ultrasonography applies sound waves and detects reflected sound for an ultrasound image.

While the present invention has been illustrated by a description of various embodiments and while these embodiments have been described in considerable detail, it is not the intention of the applicants to restrict or in any way limit the scope of the appended claims to such detail. Additional advantages and modifications will readily appear to those skilled in the art. Thus, the invention in its broader aspects is therefore not limited to the specific details, representative apparatus and method, and illustrative example shown and described. Accordingly, departures may be made from such details without departing from the spirit or scope of applicant's general inventive concept.

1. An apparatus for administering a suspendible agent in suspension, comprising:
a delivery container including a fluid reservoir capable of holding a propellant fluid, an exit port, a fluid path between said fluid reservoir and said exit port, and a delivery mechanism operative for causing said propellant fluid to flow through said fluid path; and
a suspension apparatus disposed in said fluid path, said suspension apparatus including a radial flow channel and a plurality of circumferential flow channels coupled in fluid communication by said radial flow channel, said radial flow channel and said plurality of circumferential flow channels capable of being filled with the contrast agent and in fluid communication with said exit port, wherein the contrast agent is delivered to said exit port after flowing through said radial flow channel and said plurality of circumferential flow channels when said delivery mechanism is operated to cause propellant fluid to flow through said fluid path.

2. The apparatus of claim 1 wherein said suspension apparatus further includes a plurality of circumferential dividing walls defining said plurality of circumferential flow channels.

3. The apparatus of claim 2 wherein said suspension apparatus further includes a gap formed in a corresponding one of said plurality of circumferential dividing walls that defines said radial flow channel.

4. The apparatus of claim 2 wherein said suspension apparatus includes a first plate carrying said plurality of circumferential dividing walls.

5. The apparatus of claim 4 wherein said first plate includes a radial dividing wall intersecting said plurality of circumferential dividing walls for blocking the plurality of circumferential flow channels and diverting fluid flow through said radial flow channel.

6. The apparatus of claim 4 wherein said first plate includes opposed upstream and downstream surfaces, said plurality of dividing walls being distributed between said upstream and downstream surfaces.

7. The apparatus of claim 6 wherein said first plate includes an axial flow channel coupling circumferential flow channels on said downstream surface with circumferential flow channels on said upstream surface.

8. The apparatus of claim 4 wherein said first plate includes opposed upstream and downstream surfaces and an axial flow channel extending between said upstream and downstream surfaces.

9. The apparatus of claim 8 wherein said axial flow channel is located adjacent to a center of said first plate.

10. The apparatus of claim 8 wherein said axial flow channel is located adjacent to a peripheral edge of said first plate.

11. The apparatus of claim 4 wherein said suspension apparatus further comprises a second plate contacting said plurality of first dividing walls located on said upstream surface and a third plate contacting said plurality of first dividing walls located on said upstream surface so that said plurality of first dividing walls define said plurality of circumferential flow channels.

12. The apparatus of claim 11 wherein said second and said third plates each includes an axial flow channel coupling said plurality of circumferential flow channels and said plurality of radial flow channels with circumferential and radial flow channels of an adjacent first plate.

13. The apparatus of claim 4 wherein said suspension apparatus includes a second plate having an axial flow channel communicating with said plurality of circumferential flow channels, said second plate contacting said plurality of first dividing walls for defining said plurality of circumferential flow channels.

14. The apparatus of claim 2 wherein said plurality of first dividing walls include irregularities that cause contrast agent flowing in said plurality of circumferential flow channels to change direction.

15. The apparatus of claim 2 wherein said plurality of circumferential dividing walls have a concentric arrangement.

16. The apparatus of claim 1 wherein said suspension apparatus includes a pair of first plates, said plurality of circumferential flow channels and said plurality of radial flow channels being distributed between said pair of first plates.

17. The apparatus of claim 16 wherein said suspension apparatus includes a second plate positioned between said pair of first plates so as to separate said plurality of circumferential flow channels and said plurality of radial flow channels on an upstream surface of one of said pair of first plates from said plurality of circumferential flow channels and said plurality of radial flow channels on a downstream surface of the other of said pair of first plates.

18. The apparatus of claim 17 wherein said second plate includes an axial flow channel coupling said plurality of circumferential flow channels and said plurality of radial flow channels on one of said first plates with said plurality of circumferential flow channels and said plurality of radial flow channels on the other of said first plates.

19. The apparatus of claim 1 wherein said suspension device is positioned inside said delivery container.

20. The apparatus of claim 1 wherein said circumferential flow channels have a concentric arrangement.

21. An apparatus for administering a suspendible contrast agent in suspension, comprising:
a delivery container including a fluid reservoir capable of holding a propellant fluid, an exit port, a fluid path between said fluid reservoir and said exit port, and a delivery mechanism operative for causing said propellant fluid to flow through said fluid path; and
a suspension apparatus disposed in said fluid path, said suspension apparatus including a plurality of first and second plates with a stacked arrangement, each pair of said first and second plates being separated by a plurality of dividing walls defining a plurality of circumferential flow channels capable of being filled with the contrast agent, each of said plurality of first and second plates configured to permit axial flow between said plurality of circumferential flow channels of adjacent pairs of first and second plates, wherein the contrast agent is delivered to said exit port after flowing through said plurality of circumferential flow channels when said delivery mechanism is operated to cause propellant fluid to flow through said fluid path.

22. The apparatus of claim 21 wherein a ratio of a volume of said flow channels to a volume occupied by said dividing walls ranges from about 0.25 to about 0.5.

23. The apparatus of claim 21 wherein said set of flow channels includes a concentric plurality of circumferential flow channels and a plurality of radial flow channels, adjacent pairs of said circumferential flow channels being coupled in fluid communication by a corresponding one of said radial flow channels.

24. The apparatus of claim 21 wherein each of said plurality of baffle plates and each of said plurality of spacer plates includes an axial flow channel to permit axial flow between adjacent sets of flow channels.

25. The apparatus of claim 21 wherein said suspension device is positioned inside said delivery container.

26. A method for administering a suspendible agent in suspension to a patient, comprising:
providing contrast agent in a fluid path including concentric circumferential flow channels coupled by radial flow channels and axial flow channels that confine the suspendible agent to maintain the suspension;
introducing a propellant fluid into the fluid path effective to cause the contrast agent to flow axially in the fluid path toward an exit port coupled with a patient; and
directing the suspendible agent circumferentially through the concentric circumferential flow channels and radially through the radial flow channels thereby administering the suspendible agent in suspension to the patient.

27. The method of claim 26 wherein said suspendible agent is a microbubble-containing contrast agent.

28. The method of claim 26 further comprising:
directing the contrast agent through an axial flow channel coupling adjacent sets of circumferential flow channels and radial flow channels.

29. The method of claim 26 wherein directing the contrast agent further comprises:
directing the contrast agent circumferentially through a first set of the concentric circumferential flow channels and radially outward through a first set of the radial flow channels; and
directing the contrast agent circumferentially through a second set of the concentric circumferential flow channels and radially inward through a second set of the radial flow channels

30. The method of claim 29 further comprising:
transferring the contrast agent from the first set of the concentric circumferential flow channels and the first set of the radial flow channels to the second set of the concentric circumferential flow channels and the second set of the radial flow channels.

31. The method of claim 29 further comprising:
transferring the contrast agent from the second set of the concentric circumferential flow channels and the second set of the radial flow channels to the first set of the concentric circumferential flow channels and the first set of the radial flow channels.

32. A method of filling a device for administering a suspendible agent in suspension to a patient, comprising:
aspirating a propellant fluid from a first bulk container through a fluid path including concentric circumferential flow channels coupled by radial flow channels and axial flow channels into a fluid reservoir of a delivery container; and
aspirating the suspendible agent from a second bulk container into the concentric circumferential flow channels, radial flow channels and axial flow channels of the fluid path.

33. The method of claim 32 wherein said suspendible agent is a microbubble-containing contrast agent.

34. The method of claim 32 wherein aspirating the volume of the suspendible agent further comprises:
displacing the propellant fluid resident in the concentric circumferential flow channels coupled by radial flow channels and axial flow channels into the fluid reservoir.
